(19) United States
(12) Patent Application Publication
(10) Pub. No.: US 2026/0263241 A1
(43) Pub. Date: Sep. 10, 2026

(54) IMPLANTABLE MEDICAL DEVICE WITH AN ELECTRONIC PUMP DEVICE FOR CONTROLLING A FLUIDIC DEVICE

(71) Applicant: Boston Scientific Scimed, Inc., Maple Grove, MN (US)

(72) Inventors: Brian P. Watschke, Minneapolis, MN (US); Noel Smith, Windgap (IE); Thomas Sinnott, Enniscorthy (IE); Eduardo Marcos Larangeira, Cork City (IE); Evania Ann Mareena, Clonmel (IE)

(21) Appl. No.: 19/549,721

(22) Filed: Feb. 25, 2026

Related U.S. Application Data
(60) Provisional application No. 63/766,717, filed on Mar. 4, 2025.

(51) Int. Cl. A61F 2/48; A61F 2/00; A61F 2/26

(52) U.S. Cl. CPC A61F 2/482; A61F 2/004; A61F 2/26; A61F 2/484; A61F 2250/0013

(57) ABSTRACT

According to an aspect, an implantable medical device includes an inflatable member, a fluid reservoir, and an electronic pump device. The electronic pump device includes a power converter configured to provide a voltage, a fluidic device including an actuator, a switch connected to the power converter and the actuator, and a controller configured to transition the switch between a closed state in which the power converter is connected to the actuator and an open state in which the power converter is disconnected from the actuator.

### CROSS-REFERENCE TO RELATED APPLICATION

This application claims priority to U.S. Provisional Patent Application No. 63/766,717, filed on March 4, 2025, entitled “IMPLANTABLE MEDICAL DEVICE WITH AN ELECTRONIC PUMP DEVICE FOR CONTROLLING A FLUIDIC DEVICE”, the disclosure of which is incorporated by reference herein in its entirety.

### TECHNICAL FIELD

This disclosure relates generally to an implantable medical device having an electronic pump device configured to control a fluidic device to reduce power consumption.

### BACKGROUND

Some inflatable medical devices have a pump device, which, when operated by a user, causes a transfer of fluid between a fluid reservoir and an inflatable member. The pump device may have one or more fluidic devices (e.g., pump(s) and/or valves) that uses power from a battery to transfer fluid.

### SUMMARY

This disclosure relates to an inflatable medical device with an electronic pump device configured to transfer fluid between an inflatable member and a fluid reservoir. The electronic pump device includes a power converter, a fluidic device, a switch connected to the driver and the fluidic device, and a controller configured to transition the switch between a closed state in which the power converter is connected to the fluidic device and an open state in which the power converter is disconnected from the fluidic device. In some examples, the fluidic device includes a valve (e.g., a piezo electric valve). In some examples, the fluidic device includes a pump (e.g., a piezo electric pump). When the power converter is connected to the fluidic device, a voltage (e.g., a first voltage or a second voltage) (e.g., a direct current (DC) voltage) is applied to an actuator (e.g., a piezo actuator) of the fluidic device. When the power converter is disconnected from the fluidic device, the voltage (e.g., DC voltage) is not applied to the actuator of the fluidic device. Selectively applying the voltage (e.g., the DC voltage) to the actuator of the fluidic device may increase the performance of the electronic pump device by preserving energy of the battery.

In some examples, the controller may position a valve (e.g., a piezo electric valve) in a low impedance mode applying a first voltage (e.g., a positive high DC voltage) to the valve’s actuator or position the valve in a high impedance mode by not applying the first voltage to the valve’s actuator. The controller may control the application of the first voltage to the valve’s actuator by controlling a switch that can connect or disconnect the first voltage of the power converter to the valve’s actuator. The controller may position the valve in the low impedance mode when the valve is open (e.g., during a deflation or inflation of the inflatable member), where the valve’s actuator is relatively flexible (e.g., less stiff), and the fluid path through the valve is relatively open. This may reduce the fluidic resistance leading to a more efficient operation. The controller may position the valve in the high impedance mode when the valve is closed (e.g., when maintaining pressure in the inflation member), where the valve’s actuator is relatively stiff (e.g., less flexible), thereby forming a tighter seal in the fluid chamber. The improved closure may lead to a more efficient operation.

In some examples, the controller may enable the application of a second voltage (e.g., a negative DC voltage) to a pump’s actuator in response to a deflate signal (e.g., activation of a deflation mode), which can increase the height of the fluid chamber, thereby improving passive deflation and/or reducing the high energy deflation pump operating time during active deflation. In some examples, in response to a passive deflation rate achieving a threshold level (e.g., the passive deflation rate being equal to or less than the threshold level), the controller may switch to active deflation. The threshold level may represent a switching point in terms of a threshold rate to switch from passive deflation to active deflation. Active deflation may include activating the pump to pump fluid through the pump chamber by applying a third voltage (e.g., an alternative current (AC) voltage of the power converter). Activation of the pump may cause the controller to disable the application of the second voltage (e.g., the negative DC voltage) to the pump’s actuator. The controller may control the application of the second voltage to the valve’s actuator by controlling a switch that can connect or disconnect the second voltage of the power converter to the valve’s actuator.

In some aspects, the techniques described herein relate to an implantable medical device including: an inflatable member; a fluid reservoir; and an electronic pump device configured to transfer fluid between the inflatable member and the fluid reservoir, the electronic pump device including: a power converter configured to provide a voltage; a fluidic device including an actuator; a switch connected to the power converter and the actuator; and a controller configured to transition the switch between a closed state in which the power converter is connected to the actuator and an open state in which the power converter is disconnected from the actuator.

In some aspects, the techniques described herein relate to an electronic pump device for an implantable medical device, the electronic pump device including: a power converter configured to provide a voltage; a valve; a switch connected to the power converter and the valve; and a controller configured to transition the switch between a closed state in which the power converter is connected to the valve and an open state in which the power converter is disconnected from the valve.

In some aspects, the techniques described herein relate to a method for controlling a fluidic device of an implantable medical device, the method including: detecting a first signal to open a valve to transfer fluid between an inflatable member and a fluid reservoir; in response to the first signal, applying a voltage provided by a power converter to the valve; detecting a second signal to close the valve to maintain pressure in the inflatable member; and in response to the second signal, not applying the voltage provided by the power converter to the valve.

In some aspects, the techniques described herein relate to an implantable medical device including: an inflatable member; a fluid reservoir; and an electronic pump device configured to transfer fluid between the inflatable member and the fluid reservoir, the electronic pump device including: a power converter configured to provide a voltage; a fluidic device including an actuator; a switch connected to the power converter and the actuator; and a controller configured to transition the switch between a closed state in which the power converter is connected to the actuator and an open state in which the power converter is disconnected from the actuator.

In some aspects, the techniques described herein relate to an electronic pump device for an implantable medical device, the electronic pump device including: a power converter configured to provide a voltage; a valve; a switch connected to the power converter and the valve; and a controller configured to transition the switch between a closed state in which the power converter is connected to the valve and an open state in which the power converter is disconnected from the valve.

In some aspects, the techniques described herein relate to a method for controlling a fluidic device of an implantable medical device, the method including: detecting a first signal to open a valve to transfer fluid between an inflatable member and a fluid reservoir; in response to the first signal, applying a voltage provided by a power converter to the valve; detecting a second signal to close the valve to maintain pressure in the inflatable member; and in response to the second signal, not applying the voltage provided by the power converter to the valve.

### BRIEF DESCRIPTION OF THE DRAWINGS

FIG. 1A illustrates an implantable medical device with an electrical pump device for transferring fluid between an inflatable member and a fluid reservoir according to an aspect.

FIG. 1B illustrates a circuit for selecting applying a voltage from a power converter to an actuator of a fluidic device according to an aspect.

FIG. 1C illustrates an electronic pump device for controlling a fluidic device to reduce power consumption and improve performance according to an aspect.

FIG. 1D illustrates a fluid chamber defined least in part by an actuator having an activation element and an actuator diaphragm according to an aspect.

FIG. 1E illustrates a fluid chamber with an actuator coupled to a switch according to an aspect.

FIG. 1F illustrates a deflation curve depicting changing pressure levels over time according to an aspect.

FIG. 2 illustrates an example of a pump of an inflatable medical device according to an aspect.

FIG. 3A illustrates an example of a valve of an inflatable medical device according to an aspect.

FIG. 3B illustrates an example of the valve of an inflatable medical device according to another aspect.

FIG. 4A an example of a fluidic manifold configured to attach a pump of an inflatable medical device according to an aspect.

FIG. 4B illustrates a circuit substrate coupled to the fluidic manifold according to an aspect.

FIG. 4C illustrates a perspective of the fluidic manifold according to an aspect.

FIG. 4D illustrates a perspective of the fluidic manifold according to another aspect.

FIG. 4E illustrates a perspective of a circuit substrate coupled to a fluidic manifold according to an aspect.

FIG. 5 illustrates an inflatable medical device according to an aspect.

FIG. 6 illustrates an inflatable medical device according to another aspect.

FIG. 7 illustrates an exploded view of a housing of an electronic pump device according to an aspect.

FIG. 8 illustrates a perspective of an inflatable penile prosthesis according to an aspect.

FIG. 9 illustrates an example of an artificial urinary sphincter device according to an aspect.

FIG. 10 illustrates a flowchart depicting example operations of controlling a fluidic device of an electronic pump device of an implantable medical device according to an aspect.

### DETAILED DESCRIPTION

This disclosure relates to an implantable medical device with an electronic pump device configured to transfer fluid between a fluid reservoir and an inflatable member. In some examples, the implantable medical device includes a urology device. In some examples, the implantable medical device includes a penile prosthesis with one or more inflatable cylinders. In some examples, the implantable medical device includes a urinary control device with an inflatable cuff. However, the implantable medical device may include other types of medical devices such as a stimulation device or a neuromodulation device. The electronic pump device includes one or more fluidic devices. A fluidic device may include a valve (e.g., a piezo electric valve). A fluidic device may include a pump (e.g., a piezo electric pump). The electronic pump device includes circuitry for implementing low power techniques for controlling the valve(s) and/or pump(s) to reduce energy loss and increase the performance of the valve(s) and/or pump(s).

The electronic pump device includes a power converter, a fluidic device, a switch connected to the driver and the fluidic device, and a controller configured to transition the switch between a closed state in which a voltage source of the power converter is connected to the fluidic device and an open state in which the voltage source of the power converter is disconnected from the fluidic device. When the power converter is connected to the fluidic device, a voltage (e.g., a DC voltage) is applied to an actuator of the fluidic device. When the power converter is disconnected from the fluidic device, the voltage (e.g., the DC voltage) is not applied to the actuator of the fluidic device. Selectively applying the voltage (e.g., the DC voltage) to the actuator may increase the performance of the electronic pump device by preserving energy of the battery.

In some examples, the fluidic device includes a valve. The valve may be opened to allow fluid to transfer between an inflatable member and a fluid reservoir or may be closed to prevent the transfer of fluid and/or maintain the pressure of the inflatable member or the fluid reservoir. The valve includes an actuator configured to receive an electrical signal. The actuator includes an activation element (e.g., a piezo element) and an actuator diaphragm (e.g., a flexible membrane). The actuator may form one or more sides of a fluid chamber that enables or prevents the passage of fluid. The valve may include other components such as a base plate, a ring member, etc. In some examples, the switch is connected to the actuator of the valve and the power converter. The controller is connected to the switch, where the controller can control the positioning of the switch in the open state or the closed state (e.g., by generating a control signal that instructs the switch to open or close). In some examples, when a power source (e.g., the first voltage) of the power converter is connected to the valves’ actuator (e.g., the switch is in the closed state), the power converter supplies a first voltage to the actuator of the valve. In some examples, the first voltage is a DC voltage. In some examples, the first voltage is a relatively high voltage such as a voltage in the range of 25 to 110 volts. In some examples, the first voltage is in the range of 80 to 100 volts. In some examples, the first voltage is (or around) 88 volts.

In response to the switch being in the open state, the power converter is disconnected from the actuator of the valve such that the first voltage (e.g., the DC voltage) is not applied to the actuator of the valve. In some examples, in response to the switch being in the open state, the valve operates in a high impedance mode. In the high impedance mode, the actuator is relatively stiff (e.g., less flexible), and the actuator can withstand pressure with less displacement. In some examples, in the high impedance mode, the actuator has a first stiffness level.

In response to the switch being in the closed state, the power converter is connected to the actuator of the valve such that the first voltage (e.g., the DC voltage) is applied to the valve’s actuator. In some examples, in response to the switch being in the closed state, the valve operates in a low impedance mode. In the low impedance mode, the actuator is less stiff (e.g., more flexible), and the actuator can move (e.g., move easier) in the presence of pressure. In some examples, in the low impedance mode, the actuator has a second stiffness level. The second stiffness level is less than the first stiffness level. An actuator with the second stiffness level is more flexible (e.g., less stiff) than an actuator with the first stiffness level.

In some examples, in response to a signal to open the valve to transfer the fluid through the valve (e.g., between the inflatable member and the fluid reservoir), the controller may position the switch in the closed state such that the first voltage is applied to the actuator of the valve. The controller may position the switch in the closed state by generating a control signal, which, when received at the switch, causes the switch to be in the closed state. Because the actuator is relatively softer (e.g., more flexible, less stiff) in the low impedance mode, the fluidic resistance is reduced, which can lead to a more efficient deflation or inflation operation. In some examples, the controller detects the signal to open the valve by detecting initiation of a deflate mode (e.g., detecting a deflate signal) and/or detecting initiation of an inflate mode (e.g., detecting an inflate signal).

In some examples, in response to a signal to close the valve to maintain pressure in the inflatable member or the fluid reservoir, the controller may position the switch in the open state such that the first voltage is not applied to the actuator of the valve. The controller may position the switch in the open state by generating a control signal, which, when received at the switch, causes the switch to be in the open state. Because the actuator is relatively stiffer (e.g., less flexible, more rigid) in the high impedance mode, the actuator is more tightly closed to better maintain the pressure in the inflatable member or the fluid reservoir, which may lead to a more efficient operation. In some examples, the controller detects the signal to close the valve by detecting that the pressure of the inflatable member achieves a target level (e.g., a set or target pressure level in the inflated state or a set or target pressure level in the deflated state).

In some examples, the fluidic device includes a pump. In some examples, the pump is a piezo electric pump that can actively transfer fluid between the inflatable member and the fluid reservoir. The pump includes an actuator configured to receive an electrical signal. The actuator of the pump includes an activation element (e.g., a piezo element) and an actuator diaphragm (e.g., a flexible membrane). The actuator of the pump may form one or more sides of a fluid chamber that moves fluid through the fluid chamber. The pump may include other components such as a base plate, one or more passive valve layers defining an inlet valve and/or an outlet valve, etc. In some examples, the switch is connected to the pump’s actuator and a voltage supply (e.g., a second voltage) of the power converter. In some examples, when the second voltage of the power converter is connected to the pump’s actuator (e.g., the switch is in the closed state), the power converter supplies a second voltage to the pump’s actuator. In some examples, the second voltage is a DC voltage. In some examples, the second voltage is a voltage level that is less than the first voltage. In some examples, the second voltage is a negative DC voltage. In some examples, the second voltage is in the range of -5 volts to -20 volts. In some examples, the second voltage is in the range of -10 to -15 volts. In some examples, the second voltage is -12 volts.

In response to the switch being in the open state, the power converter is disconnected from the pump’s actuator such that the second voltage (e.g., the negative DC voltage) is not applied to the pump’s actuator. In response to the switch being in the closed state, the power converter is connected to the pump’s actuator such that the second voltage is applied to the pump’s actuator.

In some examples, in response to the detection of a deflate signal, the controller positions the switch in the closed state. The controller may position the switch in the closed state by generating a control signal, which, when received at the switch, causes the switch to be in the closed state. When the second voltage is applied to the pump’s actuator, the second voltage (e.g., the negative voltage) on the pump’s actuator causes the fluid chamber height to increase, which may reduce the fluidic impedance. For example, application of the second voltage to the pump’s actuator may cause the actuator to flex upwards, thereby increasing the height of the pump (e.g., pump chamber). The increased fluid chamber height may enable the passive transfer of fluid through the pump. The passive transfer of fluid may refer to the passage of fluid through the pump without the pump being activated. The reduced resistance may improve passive deflation performance, which can reduce the high energy deflation pump operating time (e.g., reduces the amount of time spent in active deflation).

In some examples, the controller may monitor (e.g., detect, read) the pressure of the inflatable member using a pressure sensor and may compute a passive deflation rate. The passive deflation rate may be the rate of which the inflatable member deflates without activating the pump. In response to the passive deflation rate achieving a threshold level (e.g., the passive deflation rate being equal to or less than the threshold level), the controller may activate the pump to transfer fluid between the inflatable member and the fluid reservoir by applying a third voltage (e.g., an AC voltage) of the power converter. The threshold level may represent a switching point in terms of a threshold rate to switch from passive deflation to active deflation. In response to the passive deflation rate achieving the threshold level, the controller may transition from passive deflation to active deflation. Active deflation may include activating the pump to pump fluid through the pump chamber by applying a third voltage (e.g., an AC voltage) to the actuator of the pump. In response to the activation of active deflation (e.g., in response to activating the pump to transfer fluid between the inflatable member and the fluid reservoir), the controller may position the switch in the open state such that the second voltage is not applied to the pump’s actuator.

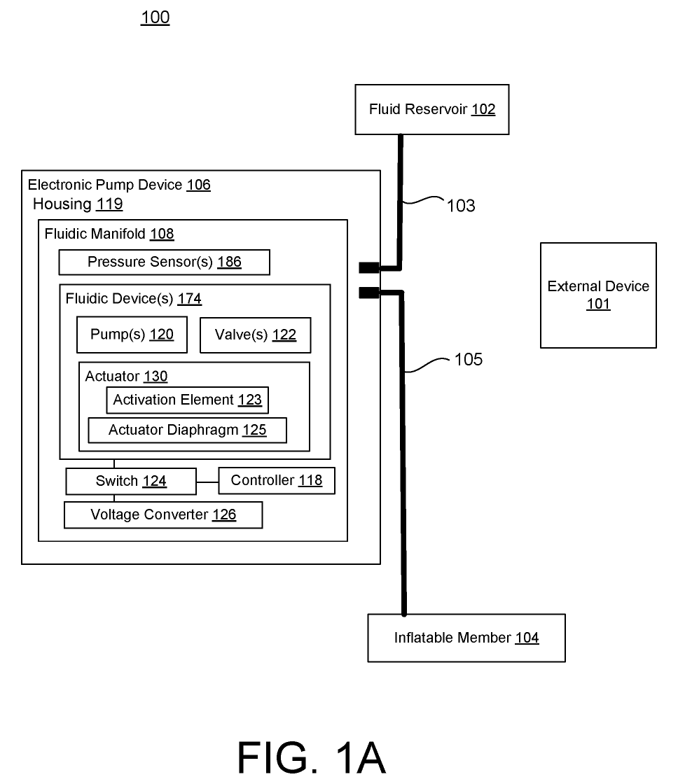

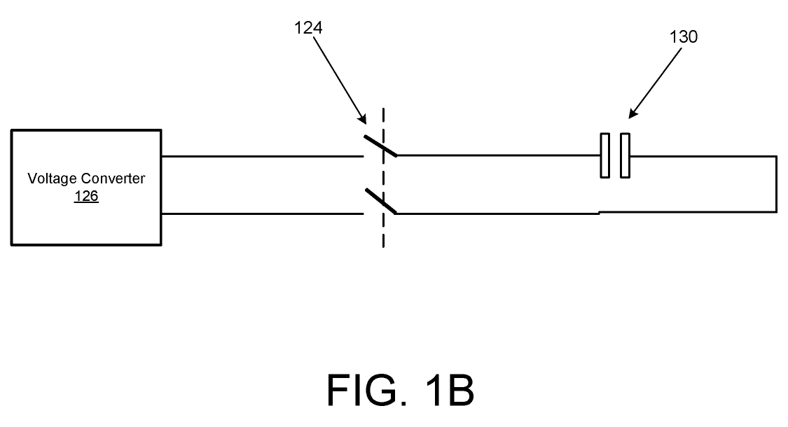

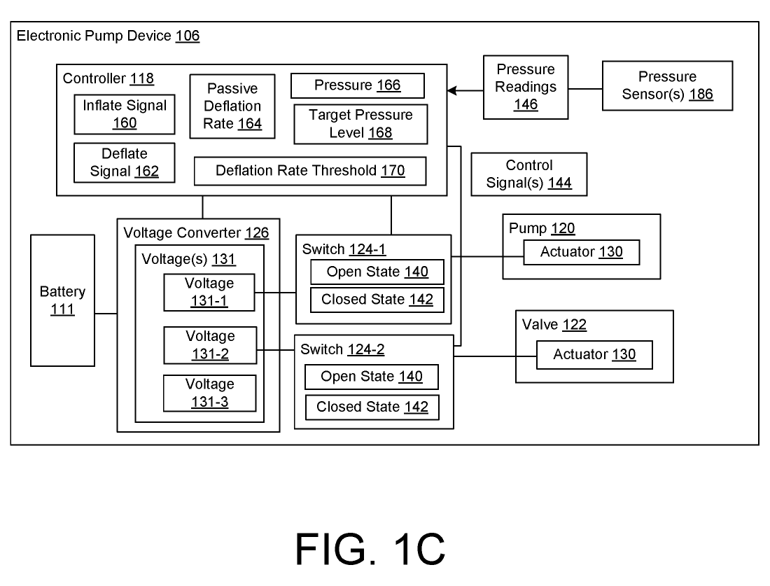

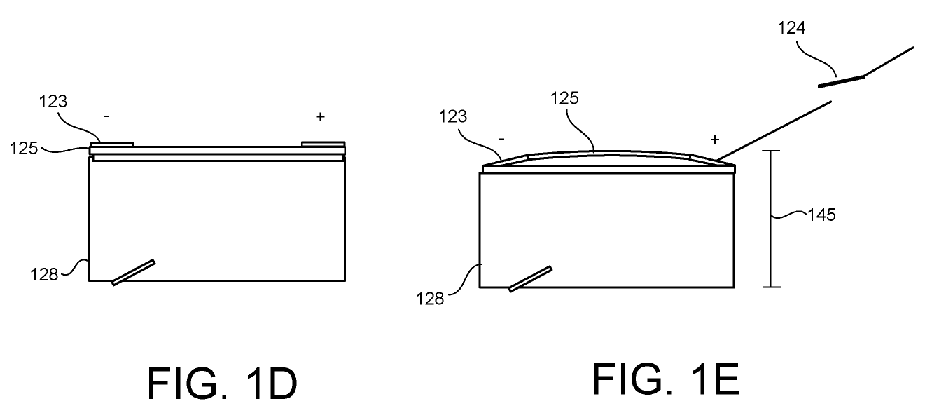

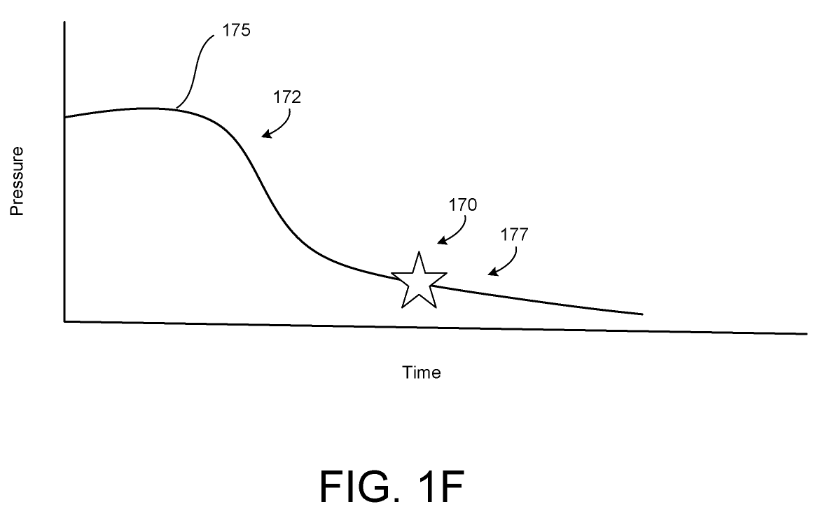

FIGS. 1A to 1F illustrate an implantable medical device 100 according to an aspect. The implantable medical device 100 configures a high impedance mode or a low impedance mode for one or more fluidic devices 174 to reduce energy consumption. The implantable medical device 100 may dynamically switch between high and low impedance modes depending on the operation. In some examples, the switching is controlled by one or more switches 124, which can alter the stiffness of the actuator 130 to improve valve performance and energy efficiency.

For example, when a switch 124 is closed, a power converter 126 supplies a voltage 131 (e.g., a voltage 131-1 or a voltage 131-2) to an actuator 130 of a fluidic device 174. This may be referred to as low impedance mode. In the low impedance mode, the actuator 130 may consume energy to maintain its position. When the valve 122 is opened for inflation, the low impedance mode may be used. In the low impedance mode, the actuator 130 is less stiff, allowing it to be pushed open relatively easily, reducing fluidic resistance, and/or facilitating efficient inflation.

However, when the switch 124 is open, the power converter 126 is disconnected from the actuator 130. This may be referred to as high impedance mode. In the high impedance mode, the actuator 130 retains its charge and position for a period of time (e.g., a significant period of time) without consuming energy from the power converter 126. The high impedance mode may be beneficial when the actuator 130 needs to maintain a static position, such as holding a valve closed. The high impedance mode may be beneficial for energy saving. By disconnecting the power converter 126, the energy consumption of the actuator 130 is reduced (e.g., significantly reduced), which is beneficial for preserving battery life of a battery 111.

In some examples, the implantable medical device 100 includes a urology device. In some examples, the implantable medical device 100 is an artificial urinary sphincter device. In some examples, the implantable medical device 100 is an inflatable penile prosthesis. However, the implantable medical device 100 may include any type of medical device that transfers fluid between components of the implantable medical device 100 such as respiratory devices, cardiovascular devices, gastrointestinal devices, ophthalmic devices, drug delivery devices, and/or diagnostic devices. In some examples, the implantable medical device 100 may be a stimulation device. In some examples, the implantable medical device 100 may be a neuromodulation device.

The implantable medical device 100 includes a fluid reservoir 102, an inflatable member 104, and an electronic pump device 106 configured to transfer fluid between the fluid reservoir 102 and the inflatable member 104. In some examples, the inflatable member 104 is an inflatable cuff member configured to be implemented around a urethra of a patient. In some examples, the inflatable member 104 is a penile inflation member (e.g., one or more inflatable cylinders) that may be implanted into the corpus cavernosum of the user. The fluid reservoir 102 may be implanted in the abdomen or pelvic cavity of the user (e.g., the fluid reservoir 102 may be implanted in the lower portion of the user’s abdominal cavity or the upper portion of the user’s pelvic cavity). In some examples, at least a portion of the electronic pump device 106 may be implemented in the patient’s body.

The inflatable member 104 may be capable of expanding upon the injection of fluid into a cavity of the inflatable member 104. If implanted around the urethra, the expansion of the inflatable member 104 causes the urethra to become restricted, thereby reducing the risk of incontinence in patients. For example, the electronic pump device 106 is configured to move fluid to pressure the inflatable cuff (e.g., the inflatable member 104), which constricts the urethra, thereby restricting the flow of urine. To urinate, the patient may operate the electronic pump device 106 to depressurize the inflatable cuff by transferring fluid from the inflatable cuff to the fluid reservoir 102. If implanted into the corpus cavernosum, upon injection of the fluid into the inflatable member 104, the inflatable member 104 may increase its length and/or width, as well as increase its rigidity.

The fluid reservoir 102 may include a container having an internal chamber configured to hold or house fluid that is used to inflate the inflatable member 104. In some examples, the fluid reservoir 102 is pressurized. In some examples, the fluid reservoir 102 is a pressurized balloon. In some examples, the implantable medical device 100 includes a single pressurized balloon. In some examples, the implantable medical device 100 includes two or more pressurized balloons. The pressure in the inflatable member 104 may be generated by the fluid reservoir 102.

The implantable medical device 100 may include a first tube member 103 and a second tube member 105. In some examples, the first tube member 103 and the second tube member 105 are referred to as conduit connectors. Each of the first tube member 103 and the second tube member 105 may define a lumen configured to transfer the fluid to and from the electronic pump device 106. The first tube member 103 may be coupled to the electronic pump device 106 and the fluid reservoir 102 such that fluid can be transferred between the electronic pump device 106 and the fluid reservoir 102 via the first tube member 103. For example, the first tube member 103 may define a first lumen configured to transfer fluid between the electronic pump device 106 and the fluid reservoir 102. The first tube member 103 may include a single or multiple tube members for transferring the fluid between the electronic pump device 106 and the fluid reservoir 102. In some examples, the first tube member 103 may be referred to as first tube members, and two first tube members can be connected together using a connector.

The second tube member 105 may be coupled to the electronic pump device 106 and the inflatable member 104 such that fluid can be transferred between the electronic pump device 106 and the inflatable member 104 via the second tube member 105. For example, the second tube member 105 may define a second lumen configured to transfer fluid between the electronic pump device 106 and the inflatable member 104. The second tube member 105 may include a single or multiple tube members for transferring the fluid between the electronic pump device 106 and the inflatable member 104. In some examples, the second tube member 105 may be referred to as second tube members, and two second tube members can be connected together using a connector. In some examples, the first tube member 103 and the second tube member 105 may include a silicone rubber material. In some examples, the electronic pump device 106 may be directly connected to the fluid reservoir 102.

The electronic pump device 106 that can monitor control and regulate the pressure within an inflatable member 104. In some examples, the electronic pump device 106 is referred to as a can. The electronic pump device 106 may automatically transfer fluid between the fluid reservoir 102 and the inflatable member 104 without the user manually operating a pump (e.g., squeezing and releasing a pump bulb). The electronic pump device 106 may include an antenna configured to wirelessly transmit (and receive) wireless signals from an external device 101. The external device 101 may be any type of component that can communicate with the electronic pump device 106. The external device 101 may be a computer, smartphone, tablet, pendant, key fob, etc. A user may use the external device 101 to control the implantable medical device 100. In some examples, the user may use the external device to inflate or deflate the inflatable member 104.

The electronic pump device 106 includes a housing 119. The housing 119 includes a fluidic manifold 108 that attaches fluid transfer and pressure regulating components. In some examples, the fluidic manifold 108 includes a conductive-based material (e.g., a metal-based material). In some examples, the fluidic manifold 108 is a titanium frame. The fluidic manifold 108 may attach a circuit substrate with electronic components such as one or more switches 124, a controller 118, and a power converter 126. The fluidic manifold 108 may attach one or more fluidic devices 174. A fluidic device 174 may be a pump 120. In some examples, a pump 120 includes an electronically controlled pump. In some examples, the pump 120 includes a piezoelectric diaphragm pump. In some examples, the fluidic manifold 108 includes a recess, and at least a portion of the pump 120 is located in the recess. A fluidic device 174 may be a valve 122. In some examples, a valve 122 includes an electronically controlled valve. In some examples, the valve 122 includes a piezo-electric diaphragm valve.

A fluidic device 174 (e.g., a pump 120 or a valve 122) includes an actuator 130. The actuator 130 may receive an electrical signal, in response to the electrical signal, the actuator 130 may deform (e.g., bend, change its shape, etc.). The actuator 130 may be one or more multiple components that are coupled together. In some examples, the actuator 130 includes one or more disc-shaped elements. In some examples, the actuator 130 includes an activation element 123 (e.g., a piezo element with one or more electrodes, a piezo disc actuator, etc.) and an actuator diaphragm 125 coupled to the activation element 123. The actuator diaphragm 125 may be a flexible membrane. In some examples, the actuator diaphragm 125 may be a metal-based flexible membrane.

In some examples, the actuator 130 includes an activation element 123 (e.g., a piezo disc actuator), an isolation layer, an adhesive material, and an actuator diaphragm 125. An electrical signal applied to the activation element 123 (e.g., the electrodes of the activation element 123) may cause the activation element 123 to deform, thereby deforming the actuator diaphragm 125 (e.g., bend, change shape). For example, when an electrical signal is applied to the electrodes of the activation element 123, the activation element 123 undergoes a physical deformation, and this deformation is transmitted to the actuator diaphragm 125, causing it to move. The movement of the actuator diaphragm 125 may open or close the valve 122 or pump fluid through a pump 120.

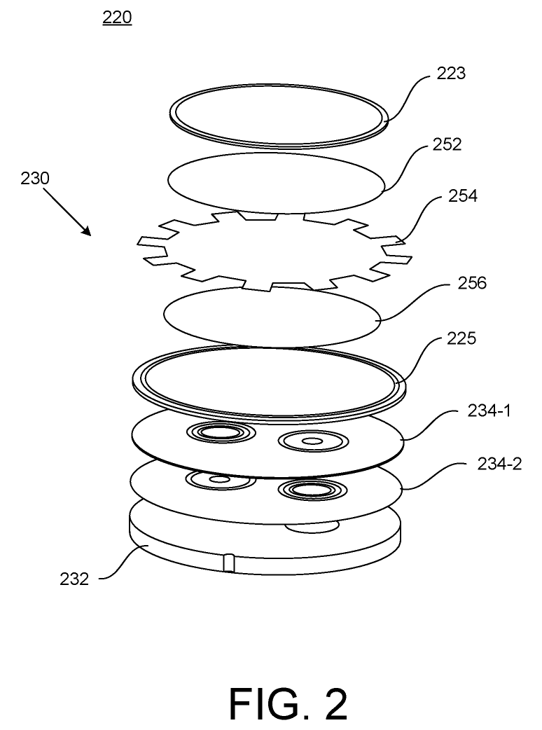

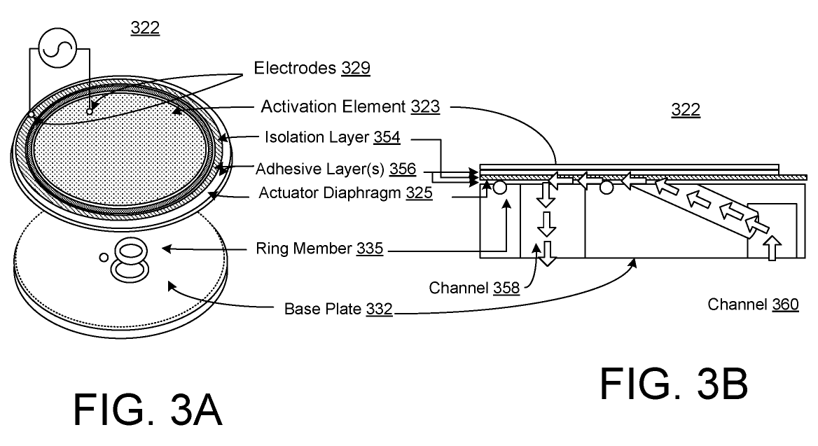

A fluidic device 174 (e.g., a pump 120 or a valve 122) may include a base plate (e.g., see base plate 232 of FIG. 2 or base plate 332 of FIGS. 3A and 3B). The base plate may be a disc-shaped metal element. The base plate may include a first hole (e.g., a first through-hole) and a second hole (e.g., a second through-hole). In some examples, the fluidic manifold 108 defines a recess (e.g., a circular opening in the frame of the fluidic manifold 108) configured to receive at least a portion of the pump 120 or the valve 122. The layers of the pump 120 or the valve 122 may form a stack, and one end of the stack may contact a surface within the recess (e.g., a recessed surface). At least a portion of the actuator 130 may be coupled (e.g., welded) to a surface (e.g., outside the recess) of the fluidic manifold 108, thereby forming a pump chamber 128.

As shown in FIG. 1A, the electronic pump device 106 includes a power converter 126 and a switch 124 connected to the power converter 126 and a fluidic device 174. The power converter 126 may provide one or more voltages 131. In some examples, the power converter 126 includes multiple voltage converters configured to generate a voltage 131-1 (e.g., DC voltage), a voltage 131-2 (e.g., DC voltage), and a voltage 131-3 (e.g., an AC voltage). In some examples, the power converter 126 includes a high voltage direct current (DC) driver, a negative DC driver, and an AC driver. A power converter 126 may be an electrical circuit that defines one or more voltage levels that are applied to a load such as the valve 122 or the pump 120. The power converter 126 is connected to a battery 111 and is configured to convert energy from the battery 111 to one or more voltages 131 (e.g., a voltage 131-1, a voltage 131-2, and a voltage 131-3). In some examples, the power converter 126 may provide two or more voltage levels, such as a voltage 131-1 and a voltage 131-2. In some examples, the voltage 131-1 and the voltage 131-2 are DC voltages. In some examples, the voltage 131-3 may be an AC voltage. The voltage 131-2 is a voltage level that is different from the voltage 131-1. In some examples, the voltage 131-1 can be selectively applied to the pump 120 (e.g., the electrodes of the activation element 123 of the pump 120), and the voltage 131-2 can be selectively applied to the valve 122 (e.g., the electrodes of the activation element 123 of the valve 122).

Application of the voltage 131-1 and the voltage 131-2 to the pump 120 and the valve 122, respectively, can be controlled by positioning one or more switches 124 in an open state 140 and a closed state 142. In some examples, as shown in FIG. 1C, the power converter 126 is connected to the pump 120 (e.g., the actuator 130 of the pump 120) via a switch 124-1. The voltage 131-1 is connected or disconnected from the pump 120 based on the state of the switch 124-1. When the switch 124-1 is in an open state 140, the power converter 126 (e.g., the voltage 131-1) is disconnected from the pump 120. When the switch 124-1 is in a closed state 142, the power converter 126 (e.g., the voltage 131-1) is connected to the pump 120. The power converter 126 is connected to the valve 122 (e.g., the actuator 130 of the valve 122) via a switch 124-2. The voltage 131-2 is connected or disconnected from the valve 122 based on the state of the switch 124-2. When the switch 124-2 is in an open state 140, the power converter 126 (e.g., the voltage 131-2) is disconnected from the valve 122. When the switch 124-2 is in a closed state 142, the power converter 126 (e.g., the voltage 131-2) is connected to the valve 122.

The controller 118 configured to transition the switch 124 (e.g., switch 124-1 or 124-2) between a closed state 142 in which the power converter 126 is connected to the fluidic device 174 (e.g., the pump 120 or the valve 122) and an open state 140 in which the power converter 126 is disconnected from the fluidic device 174. When the power converter 126 is connected to the fluidic device 174, a voltage 131 (e.g., a voltage 131-1 or a voltage 131-2) is applied to an actuator 130 of the fluidic device 174. When the power converter 126 is disconnected from the fluidic device 174, the voltage 131 is not applied to the actuator 130 of the fluidic device 174. Selectively applying the voltage 131 to the actuator 130 may increase the performance of the electronic pump device 106 by preserving energy of the battery 111.

The switch 124-2 is connected to the actuator 130 of the valve 122 and the power converter 126. The controller 118 is connected to the switch 124-2, where the controller 118 can control the positioning of the switch 124-2 in the open state 140 or the closed state 142 (e.g., by generating a control signal 144 that instructs the switch 124-2 to open or close). In some examples, when the power converter 126 is connected to the valves’ actuator 130 (e.g., the switch 124-2 is in the closed state 142), the power converter 126 supplies a voltage 131-2 to the actuator 130 of the valve 122. In some examples, the voltage 131-2 is a relatively high voltage such as a voltage in the range of 25 to 110 volts. In some examples, the voltage 131-2 is in the range of 80 to 100 volts. In some examples, the voltage 131-2 is (or around) 88 volts.

In response to the switch 124-2 being in the open state 140, the power converter 126 is disconnected from the actuator 130 of the valve 122 such that the voltage 131-2 is not applied to the actuator 130 of the valve 122. In some examples, in response to the switch 124-2 being in the open state 140, the valve 122 operates in a high impedance mode. In the high impedance mode, the actuator 130 is relatively stiff (e.g., less flexible), and the actuator 130 can withstand pressure with less displacement. In some examples, in the high impedance mode, the actuator 130 has a first stiffness level.

In response to the switch 124-2 being in the closed state 142, the power converter 126 is connected to the actuator 130 of the valve 122 such that the voltage 131-2 is applied to the valve’s actuator 130. In some examples, in response to the switch 124-2 being in the closed state 142, the valve 122 operates in a low impedance mode. In the low impedance mode, the actuator 130 is less stiff (e.g., more flexible), and the actuator 130 can move (e.g., move easier) in the presence of pressure. In some examples, in the low impedance mode, the actuator 130 has a second stiffness level. The second stiffness level is less than the first stiffness level. An actuator 130 with the second stiffness level is more flexible (e.g., less stiff) than an actuator 130 with the first stiffness level.

In some examples, in response to a signal to open the valve 122 to transfer the fluid through the valve 122 (e.g., between the inflatable member 104 and the fluid reservoir 102), the controller 118 may position the switch 124-2 in the closed state 142 such that the voltage 131-2 is applied to the actuator 130 of the valve 122. The controller 118 may position the switch 124-2 in the closed state 142 by generating a control signal 144, which, when received at the switch 124-1, causes the switch 124-2 to be in the closed state 142. Because the actuator 130 is relatively softer (e.g., more flexible, less stiff) in the low impedance mode, the fluidic resistance is reduced, which can lead to a more efficient deflation or inflation operation, as shown in FIG. 1E. In some examples, the controller 118 detects the signal to open the valve 122 by detecting initiation of a deflate mode (e.g., detecting a deflate signal 162) and/or detecting initiation of an inflate mode (e.g., detecting an inflate signal 160).

In some examples, in response to a signal to close the valve 122 to maintain pressure 166 in the inflatable member 104, the controller 118 may position the switch 124-2 in the open state 140 such that the voltage 131-2 is not applied to the actuator 130 of the valve 122. The controller 118 may position the switch 124-2 in the open state 140 by generating a control signal 144, which, when received at the switch 124-2, causes the switch 124-2 to be in the open state 140. Because the actuator 130 is relatively stiffer (e.g., less flexible, more rigid) in the high impedance mode, the actuator 130 is more tightly closed to better maintain the pressure 166 in the inflatable member 104, which may lead to a more efficient operation, as shown in FIG. 1D. In some examples, the controller 118 detects the signal to close the valve 122 by detecting that the pressure 166 of the inflatable member achieves a target pressure level 168 (e.g., a set or target pressure level in the inflated state or a set or target pressure level in the deflated state).

The electronic pump device 106 includes one or more pressure sensors 186. In some examples, the electronic pump device 106 includes a pressure sensor 186 configured to receive pressure readings 146 about the pressure 166 in the inflatable member 104. In some examples, the electronic pump device 106 includes a pressure sensor 186 configured to receive pressure readings 146 about the pressure in the fluid reservoir 102. In some examples, the controller 118 may detect, determine, or compute the pressure 166 of the inflatable member 104 based on the pressure readings 146. A target pressure level 168 may include a deflate target pressure level and an inflate target pressure level. The deflate target pressure level is the target pressure in a deflated state. The inflate target pressure level is the target pressure in an inflated state. A user or clinician may set the deflate target pressure level and/or the inflate target pressure level using an external device 101, which the electronic pump device 106 receives and stores those pressure levels as settings. Also, the user may use the external device 101 to adjust the target pressure level 168 of the inflatable member 104.

In some examples, the switch 124-1 is connected to the pump’s actuator 130 and the power converter 126. In some examples, when the power converter 126 is connected to the pump’s actuator 130 (e.g., the switch 124-1 is in the closed state 142), the power converter 126 supplies a voltage 131-1 to the pump’s actuator 130. In some examples, the voltage 131-1 is a DC voltage. In some examples, the voltage 131-1 is a voltage level that is less than the voltage 131-2. In some examples, the voltage 131-1 is a negative voltage. In some examples, the voltage 131-1 is in the range of -5 volts to -20 volts. In some examples, the voltage 131-1 is in the range of -10 to -15 volts. In some examples, the voltage 131-1 is -12 volts.

In response to the switch 124-1 being in the open state 140, the power converter 126 is disconnected from the pump’s actuator 130 such that the voltage 131-1 is not applied to the pump’s actuator 130. In response to the switch 124-1 being in the closed state 142, the power converter 126 is connected to the pump’s actuator 130 such that the voltage 131-1 is applied to the pump’s actuator 130.

In some examples, in response to the detection of a deflate signal 162, the controller 118 positions the switch 124-1 in the closed state 142. The controller 118 may position the switch 124-1 in the closed state 142 by generating a control signal 144, which, when received at the switch 124-1, causes the switch 124-1 to be in the closed state 142. When the voltage 131-1 is applied to the pump’s actuator 130, the voltage 131-1 (e.g., the negative voltage) on the pump’s actuator 130 causes a height 145 of the fluid chamber 128 to increase, which may reduce the fluidic impedance. For example, application of the voltage 131-1 to the pump’s actuator 130 may cause the actuator 130 to flex upwards, thereby increasing the height 145 of the pump 120 (e.g., pump chamber 128). The increased fluid chamber height may enable the passive transfer of fluid through the pump 120. The passive transfer of fluid may refer to the passage of fluid through the pump 120 without the pump being activated. The reduced resistance may improve passive deflation performance, which can reduce the high energy deflation pump operating time (e.g., reduces the amount of time spent in active deflation).

In some examples, the controller 118 may monitor (e.g., detect, read) the pressure 166 of the inflatable member 104 using a pressure sensor 186 and may compute a passive deflation rate 164 based on the pressure readings 146 over time. The passive deflation rate 164 may be the rate of which the inflatable member 104 deflates without activating the pump 120. In response to the passive deflation rate 164 achieving a deflation rate threshold 170 (e.g., the passive deflation rate 164 being equal to or less than the deflation rate threshold 170), the controller 118 may activate the pump 120 to transfer fluid between the inflatable member 104 and the fluid reservoir 102.

As shown in FIG. 1F, the deflation rate threshold 170 may represent a switching point in terms of a threshold rate to switch from passive deflation (at point 172) to active deflation (at point 177). For example, FIG. 1F illustrates a curve 175 with pressure 166 of the inflatable member 104 over time. When the rate of passive deflation reaches a threshold level, the controller 118 may switch to active deflation. For example, in response to the passive deflation rate 164 achieving the deflation rate threshold 170, the controller 118 may transition from passive deflation to active deflation. Active deflation may include activating the pump 120 to pump fluid through the pump chamber 128 by applying a voltage 131-3 (e.g., an AC voltage) to the actuator of the pump 120. In response to the activation of active deflation (e.g., in response to activating the pump 120 to transfer fluid between the inflatable member 104 and the fluid reservoir 102), the controller 118 may position the switch 124-1 in the open state 140 such that the voltage 131-1 is not applied to the pump’s actuator 130.

FIG. 2 illustrates an example of a pump 220 according to another aspect. The pump 220 may be a piezo electric pump. The pump 220 may be an example of the pump 120 of FIGS. 1A to 1F and may include any of the details discussed with reference to those figures. Although FIG. 2 illustrates two passive valve layers (e.g., 234-1, 234-2) between a base plate 232 and an actuator 230, the pump 220 may include one or more passive valve layers on a bottom of the base plate 232. The pump 220 may include multiple layers of disc-shaped elements.

The pump 220 includes an actuator 230. The actuator 230 includes an activation element 223 (e.g., a piezo element), an adhesive layer 252, an isolation layer 254, an adhesive layer 256, and an actuator diaphragm 225. The activation element 223 may receive an electrical signal (e.g., a voltage). In some examples, the activation element 223 and the actuator diaphragm 225 include one or more conductive (e.g., metal-based) materials. In some examples, the actuator 230 does not include an isolation layer 254. In some examples, the adhesive layer 252 includes an epoxy material. In some examples, the adhesive layer 256 includes an epoxy material. The pump 220 includes a passive valve layer 234-1, a passive valve layer 234-2, and a base plate 232. The passive valve layer 234-1 may be configured as an inlet valve, and the passive valve layer 234-2 may be configured as an outlet valve.

FIGS. 3A and 3B illustrate an example of a valve 322 according to an aspect. The valve 322 may be a piezo electric valve. The valve 322 may be an example of the valve 122 of FIGS. 1A to 1F and may include any of the details discussed with reference to those figures. The valve 322 includes an activation element 323 (e.g., a piezo element) with electrodes 329 that provide the electrical connection for applying a voltage), an isolation layer 354 that electrically isolates the activation element 323 from other elements of the valve 322, one or more adhesive layers 356, an actuator diaphragm 325, a ring member 335 (e.g., an O-ring), and a base plate 332. The actuator diaphragm 325 includes a fixable membrane that moves in response to the activation of the activation element 323, which controls the opening and closing of the valve 322.

The ring member 335 is positioned in a slot on the base plate 332 and assists with defining a fluid flow path and provides a sealing surface for the actuator diaphragm 325. The adhesive layer(s) 356 may bond the actuator diaphragm 325 to the activation element 323 and/or other components such as the isolation layer 354. The base plate 332 includes a channel 360 (e.g., an inlet channel) for receiving fluid and a channel 358 (e.g., an outlet channel) for expelling fluid from the valve 322.

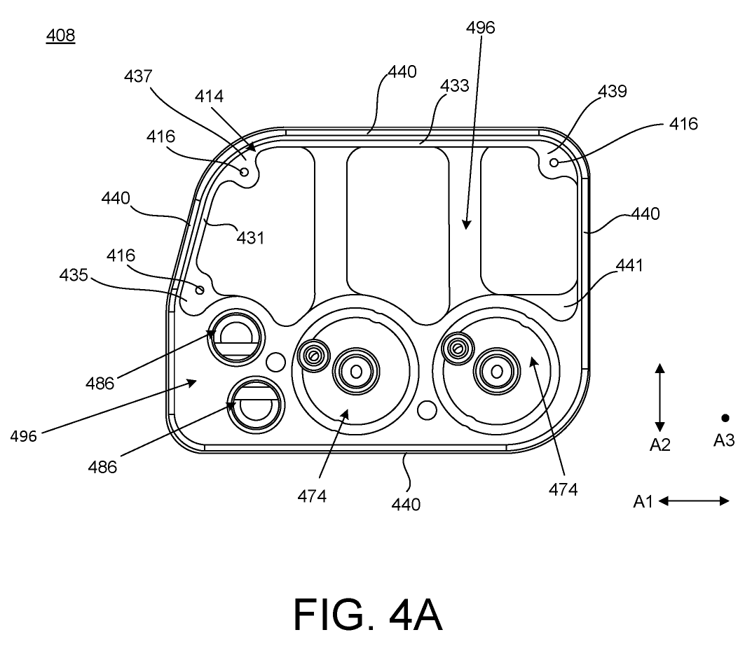

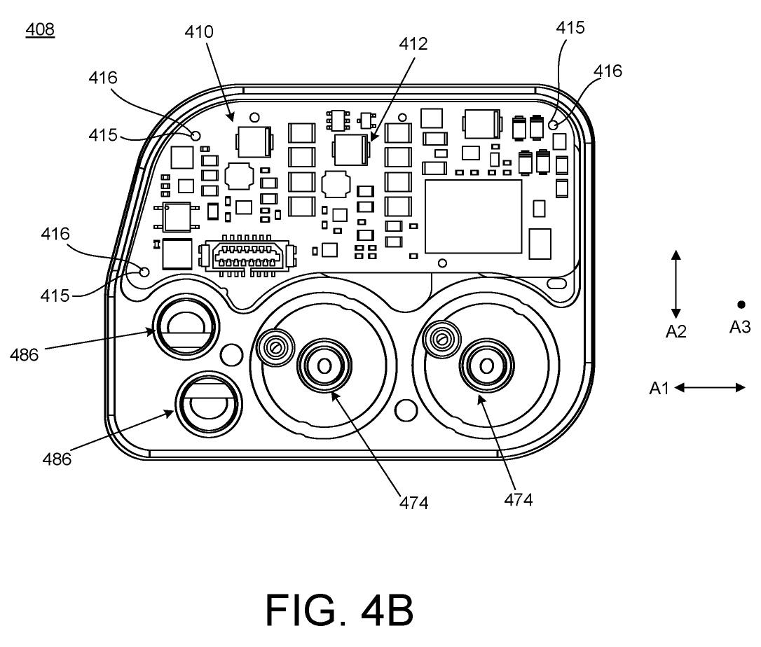

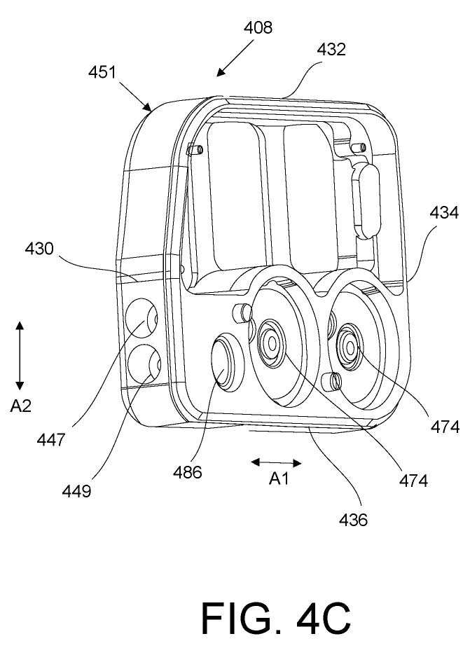

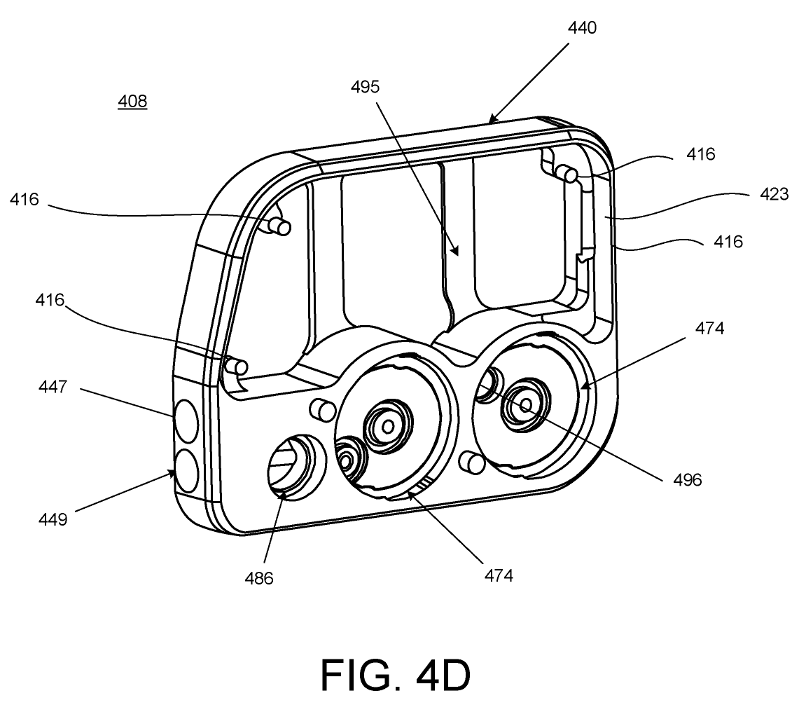

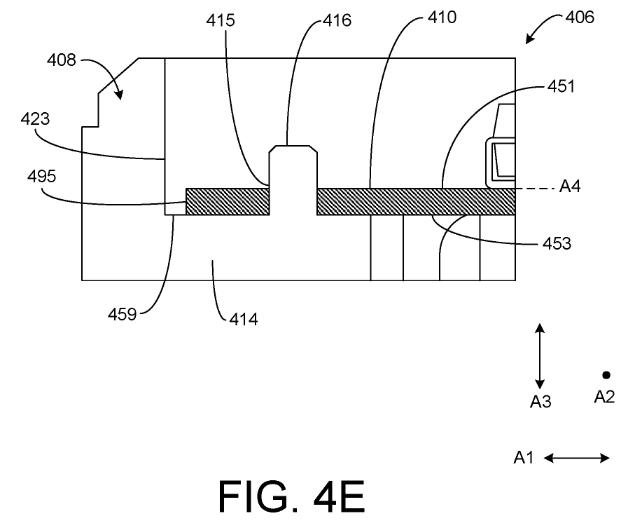

FIGS. 4A to 4E illustrates a fluidic manifold 408 of an electronic pump device 406 according to an aspect. The fluidic manifold 408 is configured to attach one or more fluidic devices 474 such as the fluidic device 174 (e.g., the pump 120, the valve 122) of FIGS. 1A to 1F, the pump 220 of FIG. 2, and/or the valve 322 of FIGS. 3A and 3B. The controller 118 of FIGS. 1A to 1F, the power converter 126 of FIGS. 1A to 1F, and the switches 124 of FIGS. 1A to 1F may be examples of electronic components 412 on a circuit substrate 710.

In some examples, the fluidic manifold 408 includes a conductive-based material (e.g., a metal-based material). In some examples, the fluidic manifold 408 is a titanium frame. The fluidic manifold 408 may attach a circuit substrate 410 with electronic components. The fluidic manifold 408 may attach one or more fluidic devices 474. A fluidic device 474 may be a pump. A fluidic device 474 may be a valve. The pump may be any of the pumps described in this disclosure. The valve may be any of the valves described in this disclosure. The fluidic manifold 408 includes a portion 496 configured to attach one or more pressure sensors 486 and one or more fluidic devices 474 (e.g., pump(s), valves). The fluidic manifold 408 includes a portion 496 configured to attach a circuit substrate 410 with electronic components 412. The electronic components 412 may include the power converter 126, the switches 124, and the controller 118 of FIGS. 1A to 1F. In some examples, the circuit substrate 410 is a printed circuit board (PCB).

In some examples, the pressure sensors 486 include a first pressure sensor connected to a fluid reservoir (e.g., the fluid reservoir 102 of FIGS. 1A to 1F) to detect the pressure in the fluid reservoir, and a second pressure sensor connected to an inflatable member (e.g., the inflatable member 104 of FIGS. 1A to 1F) to detect the pressure in the inflatable member.

The fluidic manifold 108 includes a frame 440 with an inside edge 423. The fluidic manifold 408 includes a shelf 414 that extends from the inside edge 423. The circuit substrate 410 contacts the shelf 414 (e.g., a surface 459 of the shelf 414). The circuit substrate 410 may sit on top of the shelf 414 such that the circuit substrate 410 is positioned within the frame 440. The circuit substrate 410 includes a first surface 451 and a second surface 453 that is opposite to the first surface 451. As shown in FIG. 4E, the first surface 451 of the circuit substrate 410 is disposed in the plane A4. The thickness of the circuit substrate 410 is defined as the distance between the first surface 451 and the second surface 453 in the direction A3. The circuit substrate 410 includes an edge 495. The direction A3 is orthogonal to the plane A4. The direction A1 is perpendicular to the direction A3 and perpendicular to the direction A2. The direction A2 is perpendicular to the direction A1 and perpendicular to the direction A3. The second surface 453 of the circuit substrate 410 may contact at least a portion of the surface 459 of the shelf 414.

The fluidic manifold 408 includes one or more coupling members 416 that couples the circuit substrate 410 to the fluidic manifold 408 (e.g., to the shelf 414 of the fluidic manifold 408). In some examples, the coupling members 416 includes one or more male features (e.g., protrusions, posts, fasteners, rivets, etc.) and one or more female features (e.g., holes, slots, openings, etc.). The male feature may be defined on the fluidic manifold 408 (e.g., the shelf 414 of the fluidic manifold 408) and the female features may be circuit substrate 410. In some examples, the female feature may be defined on the fluidic manifold 408 (e.g., the shelf 414 of the fluidic manifold 408) and the male features may be defined on the circuit substrate 410.

In some examples, the coupling members 416 include protrusions (e.g., posts, cylindrical posts, grooved posts, extension members, etc.) that extend from the shelf 414 in the direction A3, and the protrusions extend through holes 415 on the circuit substrate 410. In some examples, the interaction between the protrusions and the holes 415 form a press-fit coupling mechanism. Although some examples use a press-fit coupling mechanism, the circuit substrate 410, and the fluidic manifold 408 may be coupled to each other based on other types of coupling mechanisms.

The shelf 414 may include one or more shelf portions (e.g., also referred to as shoulder portions) that extend from the inside edge 423 of the frame 440 in the directions A1 and A2. The shelf 414 may have a thickness that extends in the direction A3. In some examples, the shelf portions extend from the inside edge 423 in the direction A2 and/or the direction A3 at multiple different lengths. The shelf portions may include a corner portion 435, a corner portion 437, a corner portion 439, and a corner portion 441. The corner portion 435, the corner portion 437, and the corner portion 441 may define, include, or contact a coupling member 416. The shelf portions may include a connecting portion 431 that extends between the corner portion 435 and the corner portion 437. The shelf portions may include a connecting portion 433 that extends between the corner portion 437 and the corner portion 439.

The frame 440 may define a peripheral wall formed by a wall portion 430, a wall portion 432, a wall portion 434, and a wall portion 436. In some examples, the outer surface of the wall portion 430, the wall portion 432, the wall portion 434, and the wall portion 436 form a portion of the outer surface of the electronic pump device. The fluidic manifold 408 includes a first fluid port 447 and a second fluid port 449. The first fluid port 447 and the second fluid port 449 are defined on the wall portion 430. A first tube member (e.g., tube member 103 of FIGS. 1A to 1F) may be coupled to and extend from the first fluid port 447, and a second tube member (e.g., tube member 105 of FIGS. 1A to 1F) may be coupled to and extend from the second fluid port 449. In some examples, the outer surface of the wall portion 430, the wall portion 432, the wall portion 434, and the wall portion 436 form a portion of the outer surface of the housing (e.g., the housing 119 of FIGS. 1A to 1F).

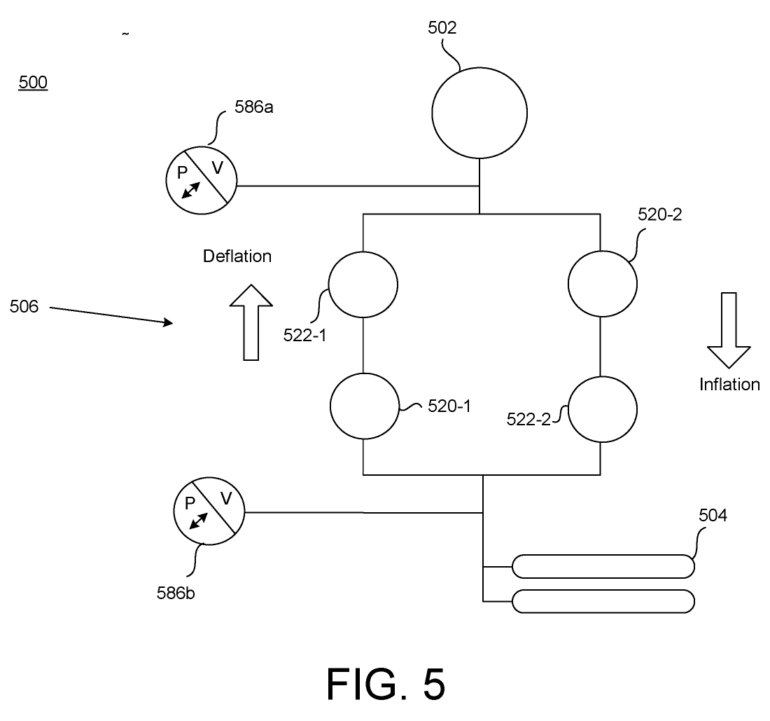

FIG. 5 illustrates an inflatable medical device 500 according to an aspect. In some examples, the implantable medical device 500 is an inflatable penile prosthesis. The implantable medical device 500 includes a fluid reservoir 502, an inflatable member 504, and an electronic pump device 506 configured to transfer fluid between the fluid reservoir 502 and the inflatable member 504 to inflate or deflate the inflatable member 504. In some examples, the inflatable member 504 is a penile inflation member (e.g., one or more inflatable cylinders) that may be implanted into the corpus cavernosum of the user.

The electronic pump device 506 includes a pressure sensor 586a connected to the fluid reservoir 502 and configured to monitor the pressure of the fluid reservoir 502, and a pressure sensor 586b connected to the inflatable member 504 and configured to monitor the pressure of the inflatable member 504.

The electronic pump device 506 includes a valve 522-1 and a pump 520-1. The valve 522-1 and the pump 520-1 are disposed in the same fluid passageway. The pump 520-1 may be an example of the pump 120 of FIGS. 1A to 1F and/or the pump 220 of FIG. 2. The valve 522-1 may be an example of the valve 122 of FIGS. 1A to 1F and/or the valve 322 of FIGS. 3A and 3B. The valve 522-1 and the pump 520-1 may be used to deflate the inflatable member 504. The electronic pump device 506 includes a pump 520-2 and a valve 522-2. The pump 520-2 and the valve 522-2 are disposed in the same fluid passageway. The pump 520-2 may be an example of the pump 120 of FIGS. 1A to 1F and/or the pump 220 of FIG. 2. The valve 522-2 may be an example of the valve 122 of FIGS. 1A to 1F and/or the valve 322 of FIGS. 3A and 3B. The valve 522-2 and the pump 520-2 may be used to inflate the inflatable member 504.

For example, in response to a deflate signal 162 (e.g., when placed in a deflation mode), a controller (e.g., the controller 118 of FIGS. 1A to 1F) may open the valve 522-1 and may activate the pump 520-1 to transfer fluid from the inflatable member 504 to the fluid reservoir 502. In the deflation mode, the valve 522-2 is closed, and the pump 520-2 is deactivated. In response to the pressure of the inflatable member 504 achieving a target deflate pressure, the controller may close the valve 522-1 and deactivate the pump 520-1. To maintain the target deflate pressure, the valve 522-1 and the valve 522-2 are closed. In response to an inflate signal 160 (e.g., when placed in an inflation mode), a controller (e.g., the controller 118 of FIGS. 1A to 1F) may open the valve 522-2 and may activate the pump 520-2. In the inflation mode, the valve 522-1 is closed, and the pump 520-1 is deactivated. In response to the pressure of the inflatable member 504 achieving a target inflate pressure, the controller may close the valve 522-2 and deactivate the pump 520-2. To maintain the target inflate pressure, the valve 522-1 and the valve 522-2 are closed. The controller (e.g., the controller 118 of FIGS. 1A to 1F) may control the valve 522-1, the pump 520-1, the pump 520-2, and/or the valve 522-2 according to power consumption reduction techniques explained with reference to FIGS. 1A to 1F.

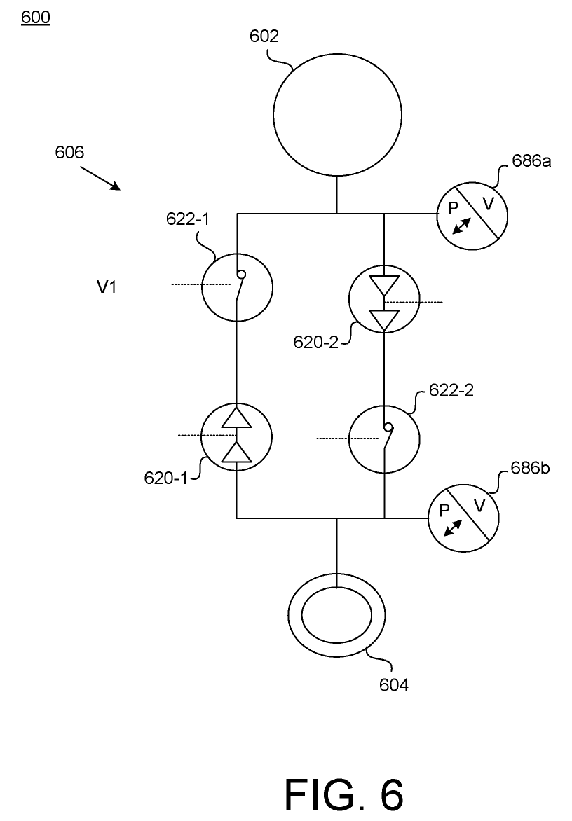

FIG. 6 illustrates an inflatable medical device 600 according to an aspect. In some examples, the implantable medical device 600 is an artificial urinary sphincter device. The implantable medical device 600 includes a fluid reservoir 602, an inflatable member 604, and an electronic pump device 606 configured to transfer fluid between the fluid reservoir 602 and the inflatable member 604 to inflate or deflate the inflatable member 604. In some examples, the inflatable member 604 is an inflatable cuff.

The electronic pump device 606 includes a pressure sensor 686a connected to the fluid reservoir 602 and configured to monitor the pressure of the fluid reservoir 602, and a pressure sensor 686b connected to the inflatable member 604 and configured to monitor the pressure of the inflatable member 604.

The electronic pump device 606 includes a valve 622-1 and a pump 620-1. The valve 622-1 and the pump 620-1 are disposed in the same fluid passageway. The pump 620-1 may be an example of the pump 120 of FIGS. 1A to 1F and/or the pump 220 of FIG. 2. The valve 622-1 may be an example of the valve 122 of FIGS. 1A to 1F and/or the valve 322 of FIGS. 3A and 3B. The valve 622-1 and the pump 620-1 may be used to deflate the inflatable member 604. The electronic pump device 606 includes a pump 620-2 and a valve 622-2. The pump 620-2 and the valve 622-2 are disposed in the same fluid passageway. The pump 620-2 may be an example of the pump 120 of FIGS. 1A to 1F and/or the pump 220 of FIG. 2. The valve 622-2 may be an example of the valve 122 of FIGS. 1A to 1F and/or the valve 322 of FIGS. 3A and 3B. The valve 622-2 and the pump 620-2 may be used to inflate the inflatable member 604.

For example, in response to a deflate signal 162 (e.g., when placed in a deflation mode), a controller (e.g., the controller 118 of FIGS. 1A to 1F) may open the valve 622-1 and may activate the pump 620-1 to transfer fluid from the inflatable member 604 to the fluid reservoir 602. In the deflation mode, the valve 622-2 is closed, and the pump 620-2 is deactivated. In response to the pressure of the inflatable member 604 achieving a target deflate pressure, the controller may close the valve 622-1 and deactivate the pump 620-1. To maintain the target deflate pressure, the valve 622-1 and the valve 622-2 are closed. In response to an inflate signal 160 (e.g., when placed in an inflation mode), a controller (e.g., the controller 118 of FIGS. 1A to 1F) may open the valve 622-2 and may activate the pump 620-2. In the inflation mode, the valve 622-1 is closed, and the pump 620-1 is deactivated. In response to the pressure of the inflatable member 604 achieving a target inflate pressure, the controller may close the valve 622-2 and deactivate the pump 620-2. To maintain the target inflate pressure, the valve 622-1 and the valve 622-2 are closed. The controller (e.g., the controller 118 of FIGS. 1A to 1F) may control the valve 622-1, the pump 620-1, the pump 620-2, and/or the valve 622-2 according to power consumption reduction techniques explained with reference to FIGS. 1A to 1F.

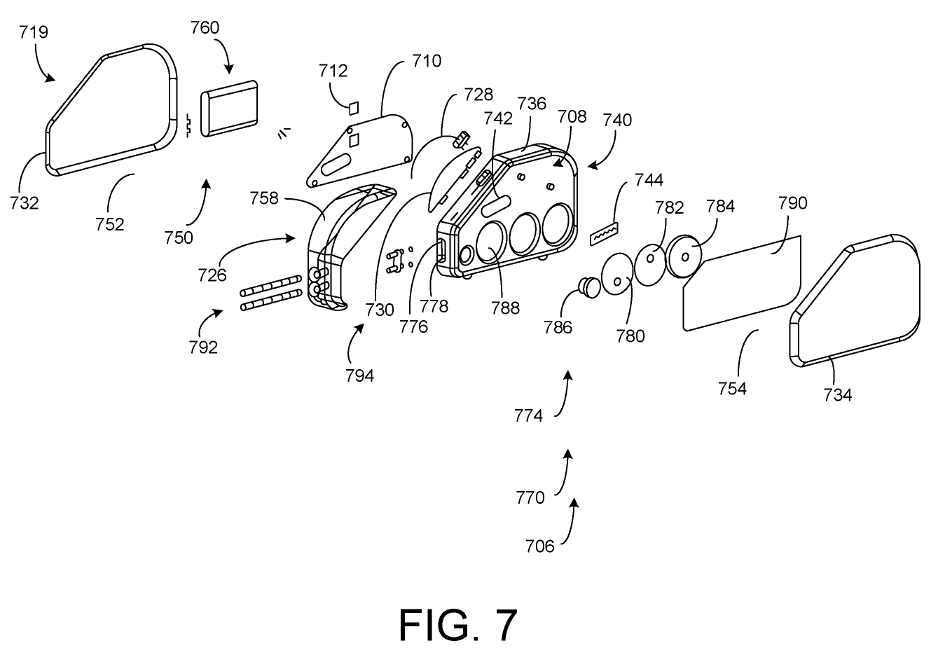

FIG. 7 illustrates an example of an exploded view of an electronic pump device 706 according to an aspect. The housing 719 includes a first sidewall 732, a second sidewall 734, a peripheral wall 736, and a frame 740. The first sidewall 732, second sidewall 734, and the peripheral wall 736 are hermetically sealed together to form an internal compartment 750 within the housing 719.

The frame 740 is disposed within the internal compartment 750 to form a first partition 752 and a second partition 754 in such a manner that the first partition 752 is hermetically sealed from the second partition 754. The frame 740 can be integrally formed with the peripheral wall 736, the first sidewall 732, and/or the second sidewall 734. In some examples, the frame 740 is welded to the peripheral wall 736 or welded to the first sidewall 732, and/or the second sidewall 734. The first sidewall 732, the peripheral wall 736, and the frame 740 may form the first partition 752. The second sidewall 734, the peripheral wall 736, and the frame 740 may form the second partition 754, which is opposite the frame 740 from the first partition 752.

The electronic pump device 706 can include a header 726 attached to the housing 719 to form an internal region 758 between an inner surface of the header 726 and an outer surface of the housing 719 that includes power and communication interface structures such as a secondary coil 728 and the antenna 730 external to the hermetically sealed housing 719. The header 726 is configured from a dielectric or insulative material, such as a radome, to allow the transmission of power and communication signals between the antenna 730 and a handset programmer or charger, and between the secondary coil 728 and the charger. For example, the header 726 may include an over-molded polymer affixed to the housing 719 and including the secondary coil 728 and the antenna 730 within the internal region 758. The secondary coil 728 and antenna 730 are constructed from a biocompatible material. In some examples, the secondary coil 728 and antenna 730 can be formed as a coil from a stamped titanium core clad with gold or silver. In some examples, the secondary coil 728 and antenna 730 can be formed from a gold wire.

The electronic pump device 706 includes an energy storage system, such as a battery (e.g., a rechargeable power source) (e.g., a rechargeable battery), and electronic components 712 within the first partition 752. The electronic components 712 may include the power converter 126, the switches 124, and the controller 118 of FIGS. 1A to 1F. The electronic components 712 can be disposed on a circuit substrate 710, such as a plurality of circuit boards, within the first partition 752. The battery 760 can assume various forms appropriate to provide power for generating desired electrical signals and to store power provided from the electronic components 712. For example, the battery 760 can incorporate lithium-ion (Li+) chemistry, e.g., a lithium-ion battery to operate the electronic components 712. In some examples, the electronic components 712 can be implemented by various components including resistors, capacitors, transistors, and integrated circuits disposed on the circuit substrate 710. The secondary coil 728 and antenna 730 are electrically coupled to the electronic components 712 within the first partition 752, such as via a hermetic feedthrough component.

The electronic components 712 can include a recharge system, a communication system, and a controller. The recharge system includes hardware configured to interface with the secondary coil 728 to receive power signals, and to provide the power signals in a form suitable to recharge the battery 760 and can include circuitry to reduce the likelihood of overcharging the battery 760. The communication system includes hardware configured to interface with the antenna 730 to receive electrical communication signals. For instance, the communication system can be configured to communicate via a wireless personal area network technology such as a short-range communication protocol (e.g., Bluetooth) (e.g., Bluetooth Low Energy), which is compatible with several operating systems that can be applied in mobile devices configured as external devices (e.g., external device 101) (e.g., handset programmers). The communication system can include an integrated circuit to implement an applied communication technology. In some examples, the communication system can be used to transmit communication signals to other devices, such as a charger or the handheld programmer (e.g., external device 101), and the communication system can be implemented to generate communication signals and provide the communication signals to the antenna 730 for transmission. In some examples, the communication system can be configured to receive and transmit radio frequency signals via the antenna 730. The controller can include a microcontroller to operate the recharge system and to receive and operate in response to communication signals or generate communication signals from the communication system.

The electronic pump device 706 also includes a fluidic circuit 770 within the second partition 754 and opposite the frame 740 from the battery 760 and electronic components 712. In some examples, the frame 740 can include an opening 742 that includes a hermetic interface 744, such as a feedthrough hermetically affixed to the frame 740. The electronic components 712 are operably coupled to the fluidic circuit 770 across the frame 740 via the hermetic interface 744. For example, the controller of the electronic components 712, powered by the battery 760, can cause the operation of the fluidic circuit 770 such as to control and monitor the fluidic circuit 770.

The fluidic circuit 770 includes a fluidic manifold 708 and fluidic devices 774 operably coupled to the fluidic manifold 708. In some examples, the fluidic manifold 708 is a structure integrated into the frame 740 such that the fluidic manifold 708 and the frame 740 together form the hermetic barrier between the first partition 752 and the second partition 754 of the internal compartment 750. For instance, the battery 760, the circuit substrate 710, or electronic components 712 can be coupled to a first major surface of the fluidic manifold 708 in the first partition 752, and the fluidic devices 774 are operably coupled to a second, and opposite major surface of the fluidic manifold 708 in the second partition 754.

The fluidic circuit 770 provides for the transfer of the fluid between the fluid reservoir (e.g., the fluid reservoir 102 of FIGS. 1 to 1F) and the inflatable member (e.g., the inflatable member 104 of FIGS. 1A to 1F). The fluidic manifold 708, which can be a hermetic manifold, segments and contains the fluid from the internal compartment 750 to reduce the chance of fluid exchange and directs the fluid from a first port 776 to a second port 778 via internal fluid passageways or channels.

The fluidic devices 774 include a plurality of fluid pumps, such as pumps 780, 782, a valve 784 mounted into the fluidic manifold 708 in fluidic communication with a manifold passageway to transfer fluid from the first port 776 to the second port 778. The pump 780 or the pump 782 may be any of the pumps described with reference to FIGS. 1A to 6. The pumps and the valve(s) are in fluid communication with a single fluid passageway between ports 776, 778. The fluidic devices 774 also includes one or more pressure sensors 786 operably coupled to the fluidic manifold 708 and in fluidic communication with the passageway to detect the pressure of the fluid within the fluidic manifold 708.

The fluidic devices 774 are included in a planar configuration on the fluidic manifold 708 in which the pumps 780, 782, valve 784, and pressure sensor 786 are mounted into the fluidic manifold 708 on a plane for slim profile within the second partition 754. The fluidic manifold 708 can include chambers 788 formed into the second major surface in which the chambers are fluidically coupled to the single passageway within the fluidic manifold 708. The chambers are configured to receive the pumps 780, 782, and valve 784 and one or more pressure sensors 786. In some examples, the fluidic manifold 708 can receive a piezoelectric pump. The fluidic manifold 708 can receive a component cover 790 over the fluidic devices 774, which can be hermetically sealed to the second major surface.

In some examples, the electronic pump device 706 may include kink resistant tubing 792 that can extend through the header 726 and attached to the ports 776, 778 via components such as a barb 794 and O-rings. The kink resistant tubing 792 can be attached to the tube members 103, 105 of FIGS. 1A to 1F to fluidically couple the electronic pump device 706 to the fluid reservoir (e.g., the fluid reservoir 102 of FIGS. 1A to 1F) and the inflatable member (e.g., the inflatable member 104 of FIGS. 1A to 1F).

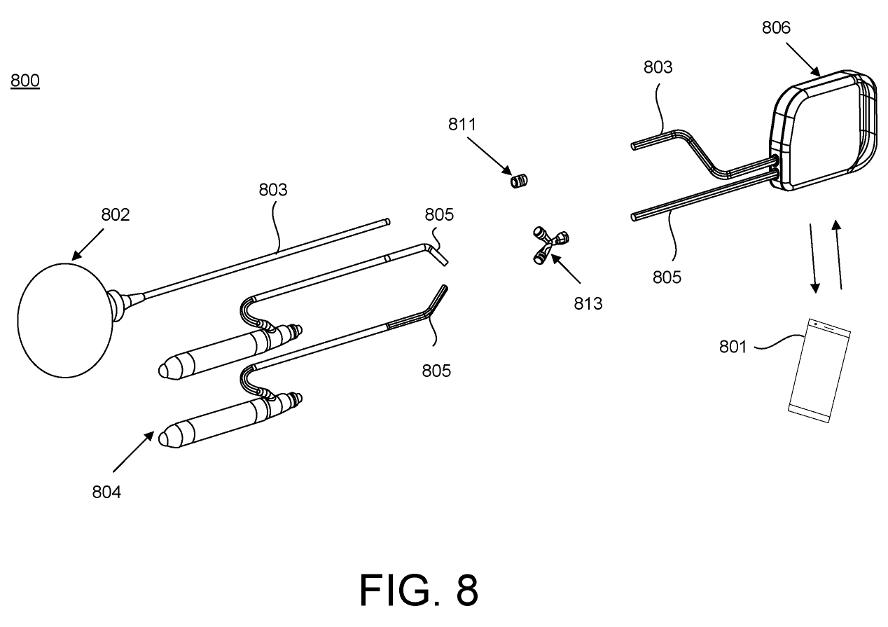

FIG. 8 illustrates a perspective of an inflatable penile prosthesis 800 according to an aspect. The inflatable penile prosthesis 800 may be an example of any of the medical devices discussed herein (e.g., including implantable medical device 100), and, therefore, may include any of the details discussed with reference to the previous figures.

The inflatable penile prosthesis 800 includes an inflatable member 804, a fluid reservoir 802, and an electronic pump device 806. The inflatable member 804 includes a pair of inflatable cylinders. The electronic pump device 806 may be an example of any of the pump devices discussed with reference to the previous figures and may include any of the details discussed herein. The electronic pump device 806 includes fluidic devices such as pumps, valves, and/or sensing devices positioned in fluid passageways. The electronic pump device 806 includes components such as, for example, one or more fluid control devices, one or more pressure sensors, and other such components. The electronic pump device 806 includes an electronic control system configured to provide for the transfer of fluid between a fluid reservoir 802 and an inflatable member 804 via the fluidic devices.

The electronic pump device 806 may include one or more integrated circuits. In some examples, the integrated circuits are included in a printed circuit board that is included in a housing of the electronic pump device 806. In some examples, the electronic pump device 806 includes the controller 118, the power converter 126, and the switches 124 of FIGS. 1A to 1F. Fluidic devices and the electronic components of the electronic pump device 806 are included in a housing. In some examples, fluidic devices and electronic components in the housing define a manifold (e.g., an electronically controlled fluidic manifold) that provides for the electronic control of the flow of fluid between the fluid reservoir 802 and the inflatable member 804. In some examples, the electronic pump device 806 can communicate with an external device 801, via respective communication modules. For example, an application stored in a memory and executed by a processor of the external device 801 may allow the user and/or a physician to operate, view, monitor and alter operation of the inflatable penile prosthesis 800.

The inflatable penile prosthesis 800 includes one or more first tube members 803 that connect a first fluid port of the electronic pump device 806 with the fluid reservoir 802. One or more second tube members 805 connect a second fluid port of the electronic pump device 806 with the inflatable member 804 in the form of the inflatable cylinders. In some examples, the inflatable penile prosthesis 800 includes a connector 811 that is used to connect two tube members 803 together, and a connector 813 that is used to connect two tube members 805 together.

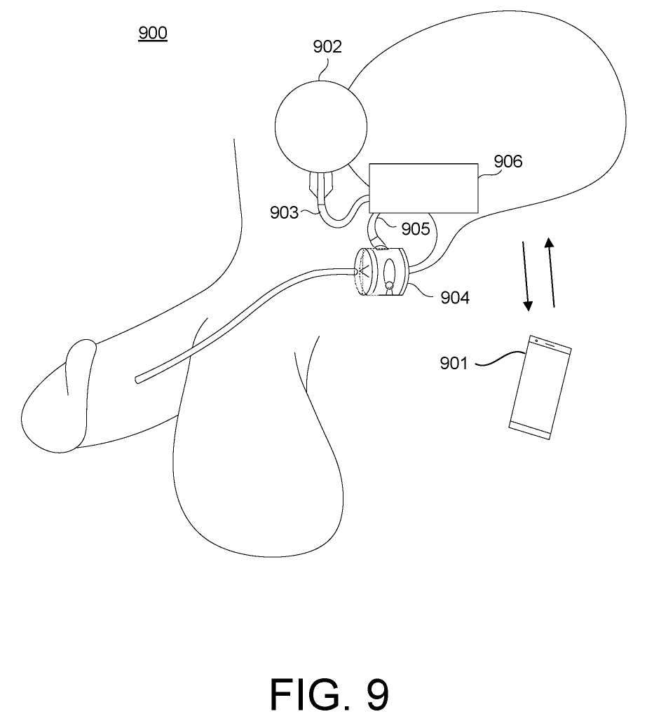

FIG. 9 illustrates a urinary control device 900 having an electronic pump device 906 according to an aspect. The urinary control device 900 may be an example of the implantable medical device 100. In some examples, the urinary control device 900 is an artificial urinary sphincter device. The electronic pump device 906 may include any of the features of the pump devices discussed herein. The urinary control device 900 includes an electronic pump device 906, a fluid reservoir 902, and a cuff 904 (e.g., an inflatable cuff). In some examples, the electronic pump device 906 includes the controller 118, the power converter 126, and the switches 124 of FIGS. 1A to 1F.

The fluid reservoir 902 may be a pressure-regulating inflation balloon or element. The fluid reservoir 902 is in operative fluid communication with the cuff 904 via one or more tube members 903, 905. The fluid reservoir 902 is constructed of polymer material that is capable of elastic deformation to reduce fluid volume within the fluid reservoir 902 and push fluid out of the fluid reservoir 902 and into the cuff 904. However, the material of the fluid reservoir 902 can be biased or include a shape memory construct adapted to generally maintain the fluid reservoir 902 in its expanded state with a relatively constant fluid volume and pressure. In some examples, this constant level of pressure exerted from the fluid reservoir 902 to the cuff 904 will keep the cuff 904 at a desired inflated state when open fluid communication is provided between the fluid reservoir 902 and the cuff 904. In some examples, the fluid reservoir 902 is implanted into the abdominal space.

A user may use an external device 901 to control the urinary control device 900. In some examples, the user may use the external device 901 to inflate or deflate the cuff 904. For example, in response to the user activating an inflation cycle using the external device 901, the external device 901 may transmit a wireless signal to the electronic pump device 906 to initiate the inflation cycle to transfer fluid from the fluid reservoir 902 to the cuff 904 (e.g., by opening an active valve where the pressure in the fluid reservoir 902 causes the fluid to move through the active valve to the cuff 904). In some examples, in response to the user activating a deflation cycle using the external device 901, the external device 901 may transmit a wireless signal to the pump device 906 to initiate the deflation cycle to transfer fluid from the cuff 904 to the fluid reservoir 902.

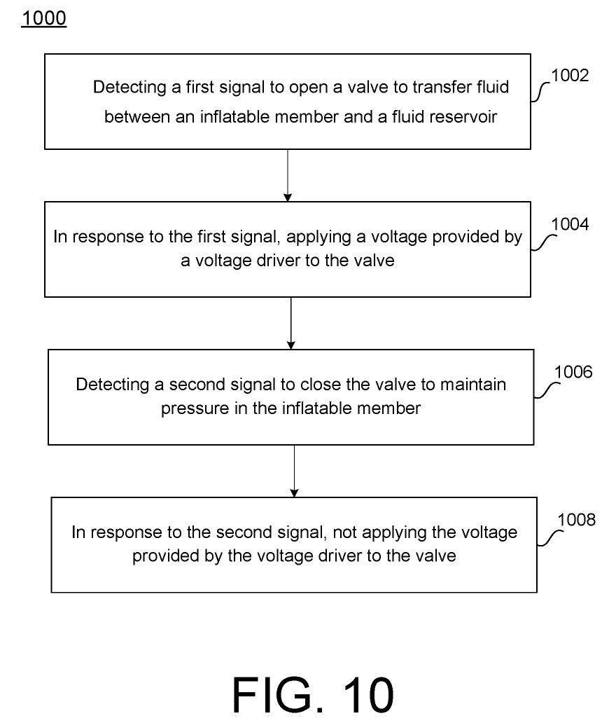

FIG. 10 illustrates a flowchart 1000 depicting example operations of controlling a fluidic device of an electronic pump device of an implantable medical device according to an aspect. Although the flowchart 1000 of FIG. 10 illustrates the operations in sequential order, it will be appreciated that this is merely an example, and that additional or alternative operations may be included. Further, operations of FIG. 10 and related operations may be executed in a different order than that shown, or in a parallel or overlapping fashion.

Operation 1002 includes detecting a first signal to open a valve to transfer fluid between an inflatable member and a fluid reservoir. Operation 1004 includes, in response to the first signal, applying a voltage provided by a power converter to the valve. Operation 1006 includes detecting a second signal to close the valve to maintain pressure in the inflatable member. Operation 1008 includes, in response to the second signal, not applying the voltage provided by the power converter to the valve.

Clause 1. An implantable medical device comprising: an inflatable member; a fluid reservoir; and an electronic pump device configured to transfer fluid between the inflatable member and the fluid reservoir, the electronic pump device including: a power converter configured to provide a voltage; a fluidic device including an actuator; a switch connected to the power converter and the actuator; and a controller configured to transition the switch between a closed state in which the power converter is connected to the actuator and an open state in which the power converter is disconnected from the actuator.

Clause 2. The implantable medical device of clause 1, wherein the fluidic device includes a valve, wherein the controller is configured to: in response to a signal to open the valve to transfer the fluid between the inflatable member and the fluid reservoir, position the switch in the closed state such that the voltage is applied to the actuator.

Clause 3. The implantable medical device of clause 1 or 2, wherein the fluidic device includes a valve, wherein the controller is configured to: in response to a signal to close the valve to maintain pressure in the inflatable member or the fluid reservoir, position the switch in the open state such that the voltage is not applied to the actuator.

Clause 4. The implantable medical device of any one of clauses 1 to 3, wherein the actuator has a first stiffness level in response to the power converter being connected to the actuator, and the actuator has a second stiffness level in response to the power converter being disconnected from the actuator, the second stiffness level being greater than the first stiffness level.

Clause 5. The implantable medical device of any one of clauses 1 to 4, further comprising: in response to a deflate signal, positioning the switch in the closed state such that the voltage is applied to the actuator.

Clause 6. The implantable medical device of clause 1, wherein the fluidic device includes a pump.

Clause 7. The implantable medical device of clause 6, wherein the voltage is a negative voltage.

Clause 8. The implantable medical device of clause 6, wherein the controller is configured to: detecting whether a passive deflation rate of the inflatable member achieves a threshold level; in response to the passive deflation rate achieving the threshold level, activating the pump to transfer fluid between from the inflatable member and the fluid reservoir; and in response to activating the pump to transfer fluid between the inflatable member and the fluid reservoir, position the switch in the open state such that the voltage is not applied to the actuator.

Clause 9. An electronic pump device for an implantable medical device, the electronic pump device comprising: a power converter configured to provide a voltage; a valve; a switch connected to the power converter and the valve; and a controller configured to transition the switch between a closed state in which the power converter is connected to the valve and an open state in which the power converter is disconnected from the valve.

Clause 10. The electronic pump device of clause 9, wherein the controller is configured to: in response to a signal to open the valve to transfer fluid between an inflatable member and a fluid reservoir, position the switch in the closed state such that the voltage is applied to the valve.

Clause 11. The electronic pump device of clause 9 or 10, wherein the controller is configured to: in response to a signal to close the valve to maintain pressure in an inflatable member or a fluid reservoir, position the switch in the open state such that the voltage is not applied to the valve; and in response to a deflate signal, position the switch in the closed state such that the voltage is applied to the valve.

Clause 12. The electronic pump device of any one of clauses 9 to 11, wherein the voltage is a first voltage, and the switch is a first switch, the electronic pump device further comprising: a pump; and a second switch connected to the pump and the power converter, the power converter configured to provide a second voltage, the controller configured to transition the second switch between a closed state in which the power converter is connected to the pump and an open state in which the power converter is disconnected from the pump, in response to the power converter being connected to the pump, the second voltage is applied to the pump.

Clause 13. A method for controlling a fluidic device of an implantable medical device, the method comprising: detecting a first signal to open a valve to transfer fluid between an inflatable member and a fluid reservoir; in response to the first signal, applying a voltage provided by a power converter to the valve; detecting a second signal to close the valve to maintain pressure in the inflatable member; and in response to the second signal, not applying the voltage provided by the power converter to the valve.

Clause 14. The method of clause 13, wherein the voltage is a first voltage, the method further comprising: detecting a deflate signal to deflate the inflatable member; and applying a second voltage provided by the power converter to a pump.

Clause 15. The method of clause 14, further comprising: detecting whether a passive deflation rate of the inflatable member achieves a threshold level; in response to the passive deflation rate achieving the threshold level, activating the pump to transfer fluid between from the inflatable member and the fluid reservoir; and in response to activating the pump to transfer fluid between the inflatable member and the fluid reservoir, not applying the second voltage to the pump.

Clause 16. An implantable medical device comprising: an inflatable member; a fluid reservoir; and an electronic pump device configured to transfer fluid between the inflatable member and the fluid reservoir, the electronic pump device including: a power converter configured to provide a voltage; a fluidic device including an actuator; a switch connected to the power converter and the actuator; and a controller configured to transition the switch between a closed state in which the power converter is connected to the actuator and an open state in which the power converter is disconnected from the actuator.

Clause 17. The implantable medical device of clause 16, wherein the fluidic device includes a valve, wherein the controller is configured to: in response to a signal to open the valve to transfer the fluid between the inflatable member and the fluid reservoir, position the switch in the closed state such that the voltage is applied to the actuator.

Clause 18. The implantable medical device of clause 16, wherein the fluidic device includes a valve, wherein the controller is configured to: in response to a signal to close the valve to maintain pressure in the inflatable member or the fluid reservoir, position the switch in the open state such that the voltage is not applied to the actuator.

Clause 19. The implantable medical device of clause 16, wherein the actuator has a first stiffness level in response to the power converter being connected to the actuator, and the actuator has a second stiffness level in response to the power converter being disconnected from the actuator, the second stiffness level being greater than the first stiffness level.

Clause 20. The implantable medical device of clause 16, further comprising: in response to a deflate signal, positioning the switch in the closed state such that the voltage is applied to the actuator.

Clause 21. The implantable medical device of clause 16, wherein the fluidic device includes a pump.

Clause 22. The implantable medical device of clause 21, wherein the voltage is a negative voltage.

Clause 23. The implantable medical device of clause 16, wherein the fluidic device includes a pump, wherein the controller is configured to: detecting whether a passive deflation rate of the inflatable member achieves a threshold level; and in response to the passive deflation rate achieving the threshold level, activating the pump to transfer fluid between from the inflatable member and the fluid reservoir.

Clause 24. The implantable medical device of clause 23, wherein the controller is configured to: in response to activating the pump to transfer fluid between the inflatable member and the fluid reservoir, position the switch in the open state such that the voltage is not applied to the actuator.

Clause 25. An electronic pump device for an implantable medical device, the electronic pump device comprising: a power converter configured to provide a voltage; a valve; a switch connected to the power converter and the valve; and a controller configured to transition the switch between a closed state in which the power converter is connected to the valve and an open state in which the power converter is disconnected from the valve.

Clause 26. The electronic pump device of clause 25, wherein the controller is configured to: in response to a signal to open the valve to transfer fluid between an inflatable member and a fluid reservoir, position the switch in the closed state such that the voltage is applied to the valve.

Clause 27. The electronic pump device of clause 25, wherein the controller is configured to: in response to a signal to close the valve to maintain pressure in an inflatable member or a fluid reservoir, position the switch in the open state such that the voltage is not applied to the valve.

Clause 28. The electronic pump device of clause 25, further comprising: in response to a deflate signal, positioning the switch in the closed state such that the voltage is applied to the valve.

Clause 29. The electronic pump device of clause 25, wherein the voltage is a first voltage, and the switch is a first switch, the electronic pump device further comprising: a pump; and a second switch connected to the pump and the power converter, the power converter configured to provide a second voltage, the controller configured to transition the second switch between a closed state in which the power converter is connected to the pump and an open state in which the power converter is disconnected from the pump, in response to the power converter being connected to the pump, the second voltage is applied to the pump.

Clause 30. The electronic pump device of clause 29, wherein the second voltage is less than the first voltage, the second voltage including a negative voltage.

Clause 31. The electronic pump device of clause 29, wherein the controller is configured to: detecting whether a passive deflation rate of an inflatable member achieves a threshold level; and in response to the passive deflation rate achieving the threshold level, activating the pump to transfer fluid between from the inflatable member and a fluid reservoir.

Clause 32. The electronic pump device of clause 31, wherein the controller is configured to: in response to activating the pump to transfer fluid between the inflatable member and a fluid reservoir, position the switch in the open state such that the second voltage is not applied to the pump.

Clause 33. A method for controlling a fluidic device of an implantable medical device, the method comprising: detecting a first signal to open a valve to transfer fluid between an inflatable member and a fluid reservoir; in response to the first signal, applying a voltage provided by a power converter to the valve; detecting a second signal to close the valve to maintain pressure in the inflatable member; and in response to the second signal, not applying the voltage provided by the power converter to the valve.

Clause 34. The method of clause 33, wherein the voltage is a first voltage, the method further comprising: detecting a deflate signal to deflate the inflatable member; and applying a second voltage provided by the power converter to a pump.

Clause 35. The method of clause 34, further comprising: detecting whether a passive deflation rate of the inflatable member achieves a threshold level; in response to the passive deflation rate achieving the threshold level, activating the pump to transfer fluid between from the inflatable member and the fluid reservoir; and in response to activating the pump to transfer fluid between the inflatable member and the fluid reservoir, not applying the second voltage to the pump.

Detailed embodiments are disclosed herein. However, it is understood that the disclosed embodiments are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the embodiments are directed to bodily implants. The term patient or user may hereafter be used for a person who benefits from the medical device or the methods disclosed in the present disclosure. For example, the patient can be a person whose body is implanted with the medical device or the method disclosed for operating the medical device by the present disclosure. For example, in some embodiments, the patient may be a human.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An implantable medical device comprising:
an inflatable member;
a fluid reservoir; and
an electronic pump device configured to transfer fluid between the inflatable member and the fluid reservoir, the electronic pump device including:
a power converter configured to provide a voltage;
a fluidic device including an actuator;
a switch connected to the power converter and the actuator; and
a controller configured to transition the switch between a closed state in which the power converter is connected to the actuator and an open state in which the power converter is disconnected from the actuator.

2. The implantable medical device of claim 1, wherein the fluidic device includes a valve, wherein the controller is configured to:
in response to a signal to open the valve to transfer the fluid between the inflatable member and the fluid reservoir, position the switch in the closed state such that the voltage is applied to the actuator.

3. The implantable medical device of claim 1, wherein the fluidic device includes a valve, wherein the controller is configured to:
in response to a signal to close the valve to maintain pressure in the inflatable member or the fluid reservoir, position the switch in the open state such that the voltage is not applied to the actuator.

4. The implantable medical device of claim 1, wherein the actuator has a first stiffness level in response to the power converter being connected to the actuator, and the actuator has a second stiffness level in response to the power converter being disconnected from the actuator, the second stiffness level being greater than the first stiffness level.

5. The implantable medical device of claim 1, further comprising:
in response to a deflate signal, positioning the switch in the closed state such that the voltage is applied to the actuator.

6. The implantable medical device of claim 1, wherein the fluidic device includes a pump.

7. The implantable medical device of claim 6, wherein the voltage is a negative voltage.

8. The implantable medical device of claim 1, wherein the fluidic device includes a pump, wherein the controller is configured to:
detect whether a passive deflation rate of the inflatable member achieves a threshold level; and
in response to the passive deflation rate achieving the threshold level, activating the pump to transfer fluid between from the inflatable member and the fluid reservoir.

9. The implantable medical device of claim 8, wherein the controller is configured to:
in response to activating the pump to transfer fluid between the inflatable member and the fluid reservoir, position the switch in the open state such that the voltage is not applied to the actuator.

10. An electronic pump device for an implantable medical device, the electronic pump device comprising:
a power converter configured to provide a voltage;
a valve;
a switch connected to the power converter and the valve; and
a controller configured to transition the switch between a closed state in which the power converter is connected to the valve and an open state in which the power converter is disconnected from the valve.

11. The electronic pump device of claim 10, wherein the controller is configured to:
in response to a signal to open the valve to transfer fluid between an inflatable member and a fluid reservoir, position the switch in the closed state such that the voltage is applied to the valve.

12. The electronic pump device of claim 10, wherein the controller is configured to:
in response to a signal to close the valve to maintain pressure in an inflatable member or a fluid reservoir, position the switch in the open state such that the voltage is not applied to the valve.

13. The electronic pump device of claim 10, further comprising:
in response to a deflate signal, positioning the switch in the closed state such that the voltage is applied to the valve.

14. The electronic pump device of claim 10, wherein the voltage is a first voltage, and the switch is a first switch, the electronic pump device further comprising:
a pump; and
a second switch connected to the pump and the power converter,
the power converter configured to provide a second voltage,
the controller configured to transition the second switch between a closed state in which the power converter is connected to the pump and an open state in which the power converter is disconnected from the pump, in response to the power converter being connected to the pump, the second voltage is applied to the pump.

15. The electronic pump device of claim 14, wherein the second voltage is less than the first voltage, the second voltage including a negative voltage.

16. The electronic pump device of claim 14, wherein the controller is configured to:
detect whether a passive deflation rate of an inflatable member achieves a threshold level; and
in response to the passive deflation rate achieving the threshold level, activating the pump to transfer fluid between from the inflatable member and a fluid reservoir.

17. The electronic pump device of claim 16, wherein the controller is configured to:
in response to activating the pump to transfer fluid between the inflatable member and a fluid reservoir, position the switch in the open state such that the second voltage is not applied to the pump.

18. A method for controlling a fluidic device of an implantable medical device, the method comprising:
detecting a first signal to open a valve to transfer fluid between an inflatable member and a fluid reservoir;
in response to the first signal, applying a voltage provided by a power converter to the valve;
detecting a second signal to close the valve to maintain pressure in the inflatable member; and
in response to the second signal, not applying the voltage provided by the power converter to the valve.

19. The method of claim 18, wherein the voltage is a first voltage, the method further comprising:
detecting a deflate signal to deflate the inflatable member; and
applying a second voltage provided by the power converter to a pump.

20. The method of claim 19, further comprising:
detecting whether a passive deflation rate of the inflatable member achieves a threshold level;
in response to the passive deflation rate achieving the threshold level, activating the pump to transfer fluid between from the inflatable member and the fluid reservoir; and
in response to activating the pump to transfer fluid between the inflatable member and the fluid reservoir, not applying the second voltage to the pump.
